SEQUENCE LISTING (34 sequences)

SEQ ID NO: 1 - protein, 21 aa, Artificial Sequence
  Synthetic Construct
MALPVTALLLPLALLLHAARP

SEQ ID NO: 2 - protein, 10 aa, Artificial Sequence
  Synthetic Construct
EQKLISEEDL

SEQ ID NO: 3 - protein, 12 aa, Artificial Sequence
  Synthetic Construct
ESKYGPPCPPCP

SEQ ID NO: 4 - protein, 28 aa, Artificial Sequence
  Synthetic Construct
MFWVLVVVGGVLACYSLLVTVAFIIFWV

SEQ ID NO: 5 - protein, 41 aa, Artificial Sequence
  Synthetic Construct
RSKRSRLLHSDYMNMTPRRPGPTRKHYQPYAPPRDFAAYRS

SEQ ID NO: 6 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 19 aa, Artificial Sequence
  Synthetic Construct
ATNFSLLKQAGDVEENPGP

SEQ ID NO: 8 - protein, 236 aa, Artificial Sequence
  Synthetic Construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 9 - protein, 126 aa, Artificial Sequence
  Synthetic Construct
QVQLQESGGGLVQPGGSLRLSCAASGYPYGYTFSSYCMRWFRQAPGKDREGVARFERNGLTTYYDDSVKGRFTISQDNVKNTVYLQMNSLKPEDTATYYCAAAPKQLRTCGDYNYWGQGTQVTVSS

SEQ ID NO: 10 - protein, 123 aa, Artificial Sequence
  Synthetic Construct
QVQLQESGGGSVQAGGSLRLSCAVSGSSYSRFRMGWFRQVPGKEREGVAAIYRSDGRTYYADSVKGRFTISQDNTKNTVYLQMNSLKPEDTAMYYCAAGAYSSYLMDANFAYWGQGTQVTVSS

SEQ ID NO: 11 - protein, 130 aa, Artificial Sequence
  Synthetic Construct
QVQLQESGGGSVQAGGSLRLSCAASSYTYSYSCMAWFRQAPGKERERVASIHTGTGTANYADSAKGRFTISQDIAANTVYLQMNSLKLEDTAMYYCAARPGSAALRCTTDYSKPHDFTYWGQGTQVTVSS

SEQ ID NO: 12 - protein, 119 aa, Artificial Sequence
  Synthetic Construct
QVQLQESGGGLVQPGGSLRLSCAASGFTFSSNYMTWVRQAPGKGPEWVSGINPDGSSTYYADSVKGRFTISRDNAKNTLYLQMNSLKSEDTALYKCATGAAPRIPTTLRGQGTQVTVSS

SEQ ID NO: 13 - protein, 12 aa, Artificial Sequence
  Synthetic Construct
GYPYGYTFSSYC

SEQ ID NO: 14 - protein, 8 aa, Artificial Sequence
  Synthetic Construct
FERNGLTT

SEQ ID NO: 15 - protein, 15 aa, Artificial Sequence
  Synthetic Construct
AAAPKQLRTCGDYNY

SEQ ID NO: 16 - protein, 8 aa, Artificial Sequence
  Synthetic Construct
GSSYSRFR

SEQ ID NO: 17 - protein, 8 aa, Artificial Sequence
  Synthetic Construct
IYRSDGRT

SEQ ID NO: 18 - protein, 16 aa, Artificial Sequence
  Synthetic Construct
AAGAYSSYLMDANFAY

SEQ ID NO: 19 - protein, 8 aa, Artificial Sequence
  Synthetic Construct
SYTYSYSC

SEQ ID NO: 20 - protein, 8 aa, Artificial Sequence
  Synthetic Construct
IHTGTGTA

SEQ ID NO: 21 - protein, 23 aa, Artificial Sequence
  Synthetic Construct
AARPGSAALRCTTDYSKPHDFTY

SEQ ID NO: 22 - protein, 8 aa, Artificial Sequence
  Synthetic Construct
GFTFSSNY

SEQ ID NO: 23 - protein, 8 aa, Artificial Sequence
  Synthetic Construct
INPDGSST

SEQ ID NO: 24 - protein, 12 aa, Artificial Sequence
  Synthetic Construct
ATGAAPRIPTTL

SEQ ID NO: 25 - protein, 193 aa, Artificial Sequence
  Synthetic Construct
ESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 8 aa, Artificial Sequence
  Synthetic Construct
WSHPQFEK

SEQ ID NO: 27 - protein, 339 aa, Artificial Sequence
  Synthetic Construct
  VARIANT (127)..(146): Xaa = Can be absent or present
  VARIANT (127)..(146): Xaa = Any Amino Acid
QVQLQESGGGLVQPGGSLRLSCAASGYPYGYTFSSYCMRWFRQAPGKDREGVARFERNGLTTYYDDSVKGRFTISQDNVKNTVYLQMNSLKPEDTATYYCAAAPKQLRTCGDYNYWGQGTQVTVSSXXXXXXXXXXXXXXXXXXXXESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 336 aa, Artificial Sequence
  Synthetic Construct
  VARIANT (124)..(143): Xaa = Can be absent or present
  VARIANT (124)..(143): Xaa = Any Amino Acid
QVQLQESGGGSVQAGGSLRLSCAVSGSSYSRFRMGWFRQVPGKEREGVAAIYRSDGRTYYADSVKGRFTISQDNTKNTVYLQMNSLKPEDTAMYYCAAGAYSSYLMDANFAYWGQGTQVTVSSXXXXXXXXXXXXXXXXXXXXESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 343 aa, Artificial Sequence
  Synthetic Construct
  VARIANT (131)..(150): Xaa = Can be absent or present
  VARIANT (131)..(150): Xaa = Any Amino Acid
QVQLQESGGGSVQAGGSLRLSCAASSYTYSYSCMAWFRQAPGKERERVASIHTGTGTANYADSAKGRFTISQDIAANTVYLQMNSLKLEDTAMYYCAARPGSAALRCTTDYSKPHDFTYWGQGTQVTVSSXXXXXXXXXXXXXXXXXXXXESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 332 aa, Artificial Sequence
  Synthetic Construct
  VARIANT (120)..(139): Xaa = Can be absent or present
  VARIANT (120)..(139): Xaa = Any Amino Acid
QVQLQESGGGLVQPGGSLRLSCAASGFTFSSNYMTWVRQAPGKGPEWVSGINPDGSSTYYADSVKGRFTISRDNAKNTLYLQMNSLKSEDTALYKCATGAAPRIPTTLRGQGTQVTVSSXXXXXXXXXXXXXXXXXXXXESKYGPPCPPCPMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 865 aa, Homo sapiens
MASLYQRFTGKINTSRSFPAPPEASHLLGGQGPEEDGGAGAKPLGPRAQAAAPRERGGGGGGAGGRPRFQYQARSDGDEEDELVGSNPPQRNWKGIAIALLVILVICSLIVTSVILLTPAEDNSLSQKKKVTVEDLFSEDFKIHDPEAKWISDTEFIYREQKGTVRLWNVETNTSTVLIEGKKIESLRAIRYEISPDREYALFSYNVEPIYQHSYTGYYVLSKIPHGDPQSLDPPEVSNAKLQYAGWGPKGQQLIFIFENNIYYCAHVGKQAIRVVSTGKEGVIYNGLSDWLYEEEILKTHIAHWWSPDGTRLAYAAINDSRVPIMELPTYTGSIYPTVKPYHYPKAGSENPSISLHVIGLNGPTHDLEMMPPDDPRMREYYITMVKWATSTKVAVTWLNRAQNVSILTLCDATTGVCTKKHEDESEAWLHRQNEEPVFSKDGRKFFFIRAIPQGGRGKFYHITVSSSQPNSSNDNIQSITSGDWDVTKILAYDEKGNKIYFLSTEDLPRRRQLYSANTVGNFNRQCLSCDLVENCTYFSASFSHSMDFFLLKCEGPGVPMVTVHNTTDKKKMFDLETNEHVKKAINDRQMPKVEYRDIEIDDYNLPMQILKPATFTDTTHYPLLLVVDGTPGSQSVAEKFEVSWETVMVSSHGAVVVKCDGRGSGFQGTKLLHEVRRRLGLLEEKDQMEAVRTMLKEQYIDRTRVAVFGKDYGGYLSTYILPAKGENQGQTFTCGSALSPITDFKLYASAFSERYLGLHGLDNRAYEMTKVAHRVSALEEQQFLIIHPTADEKIHFQHTAELITQLIRGKANYSLQIYPDESHYFTSSSLKQHLYRSIINFFVECFRIQDKLLTVTAKEDEEED

SEQ ID NO: 32 - protein, 45 aa, Artificial Sequence
  Synthetic Construct
TTTPAPRPPTPAPTIASQPLSLRPEACRPAAGGAVHTRGLDFACD

SEQ ID NO: 33 - protein, 24 aa, Artificial Sequence
  Synthetic Construct
IYIWAPLAGTCGVLLLSLVITLYC

SEQ ID NO: 34 - protein, 39 aa, Artificial Sequence
  Synthetic Construct
IEVMYPPPYLDNEKSNGTIIHVKGKHLCPSPLFPGPSKP

(19) United States
(12) Patent Application Publication
(10) Pub. No.: US 2023/0381228 A1
(43) Pub. Date: Nov. 30, 2023

(54) ANTI-DPP6 CHIMERIC ANTIGEN RECEPTOR BEARING REGULATORY T CELLS

(71) Applicants: The Regents of the University of California, Oakland, CA (US); Université Libre de Bruxelles, Brussels (BE); Vrije Universiteit Brussel, Brussels (BE)

(72) Inventors: Qizhi TANG, San Francisco, CA (US); Jeffrey A. BLUESTONE, San Francisco, CA (US); Emilie RONIN, San Francisco, CA (US); Decio L. EIZIRIK, Ditbeek (BE); Nick M. DEVOOGDT, Eppegem (BE)

(73) Assignees: The Regents of the University of California, Oakland, CA (US); Université Libre de Bruxelles, Brussels (BE); Vrije Universiteit Brussel, Brussels (BE)

(21) Appl. No.: 18/034,037

(22) PCT Filed: Oct. 29, 2021
(86) PCT No.: PCT/US21/72139

§ 371 Date: Apr. 26, 2023

Related U.S. Application Data
(60) Provisional application No. 63/107,110, filed on Oct. 29, 2020.

(51) Int. Cl. A61K 35/17; C07K 14/725; C12N 5/0783; A61P 3/10; C07K 16/28; C07K 14/705; A61K 39/00

(52) U.S. Cl. CPC A61K 35/17; C07K 14/7051; C12N 5/0636; A61P 3/10; C07K 16/2812; C07K 14/70521; A61K 39/4631

(57) ABSTRACT

The present disclosure relates generally to regulatory T cells (Tregs) engineered to express a dipeptidyl aminopeptidase-like protein 6 (DPP6)-reactive chimeric antigen receptor (CAR). The engineered Tregs are suitable for use in immunotherapy regimens for autoimmune, inflammatory and degenerative diseases. In particular, the anti-DPP6 CAR expressing Tregs are suitable for treating or preventing autoimmune diseases of the pancreas or central nervous system.

### CROSS-REFERENCE TO RELATED APPLICATIONS

This application claims benefit of U.S. Provisional Application No. 63/107,110, filed Oct. 29, 2020, the disclosure of which is hereby incorporated by reference in its entirety.

### STATEMENT REGARDING FEDERALLY SPONSORED RESEARCH OR DEVELOPMENT

This invention was made with government support under Grant No. UC4 DK116264 awarded by the National Institute of Diabetes and Digestive and Kidney Diseases. The government has certain rights in the invention.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 643662002740SEQLIST.TXT, date recorded: Oct. 29, 2021, size: 33,738 bytes).

### FIELD

The present disclosure relates generally to regulatory T cells (Tregs) engineered to express a dipeptidyl aminopeptidase-like protein 6 (DPP6)-reactive chimeric antigen receptor (CAR). The engineered Tregs are suitable for use in immunotherapy regimens for autoimmune, inflammatory and degenerative diseases.

### BACKGROUND

Autoimmune diseases are a diverse collection of diseases arising as a consequence of attacks on one or more organs by an acquired immune response of an individual (e.g., autoantibody-mediated or self-reactive T cell-mediated). Autoimmune diseases are typically classified as systemic or organ-specific. Traditional treatments include immunosuppressants, such as non-steroidal anti-inflammatory drugs and glucocorticoids, which are administered to lessen the autoimmune response. Other treatments are administered to supplement or replace an organ-specific deficiency, but do not cure the underlying autoimmune diseases. For instance, diabetes mellitus can be well managed by subcutaneous injection of insulin. However, insulin injections do not achieve the tight glucose control of pancreatic islets, and therefore insulin therapy poses risks of complications from hyper- and hypoglycemia.

Thus, what is needed in the art are therapies for autoimmune diseases that are directed to inhibiting the underlying autoimmune response.

### BRIEF SUMMARY

The present disclosure relates generally to regulatory T cells (Tregs) engineered to express a dipeptidyl aminopeptidase-like protein 6 (DPP6)-reactive chimeric antigen receptor (CAR). The engineered Tregs are suitable for use in immunotherapy regimens for autoimmune, inflammatory and degenerative diseases. In particular, the anti-DPP6 CAR expressing Tregs are suitable for treating or preventing autoimmune diseases of the pancreas or central nervous system.

### BRIEF DESCRIPTION OF THE DRAWINGS

FIG. 1 shows nanobody binding to primary human islets as determined using flow cytometry.

FIG. 2A shows a schematic diagram of a nanobody-based chimeric antigen receptor (CAR). FIG. 2B shows a schematic diagram of an anti-human leukocyte antigen (HLA)-A2 single-chain variable fragment (scFv)-based CAR. In an exemplary anti-HLA-A2 CAR, the tag is a strep tag (WSHPQFEK set forth as SEQ ID NO:26). FIG. 2C shows a schematic diagram if an anti-dipeptidyl aminopeptidase-like protein 6 (DPP6) nanobody-based CAR. In an exemplary anti-DPP6 CAR, the tag is a myc tag (EQKLISEEDL set forth as SEQ ID NO:2).

FIGS. 3A-3B show anti-DPP6 CAR expression on primary human CD4+ T cells.

FIG. 3A shows anti-DPP6 CAR expression on conventional T cells (Tconvs). FIG. 3B shows anti-DPP6 CAR expression on regulatory T cells (Tregs).

FIGS. 4A-4F show activation of CD4+ Tconvs by human islets in vitro. Tconvs were cultured with or without dissociated islet cells from an HLA-A2-positive donor or an HLA-A2-negative donor, and expression of the activation markers CD71, ICOS, and CD25 was measured using flow cytometry. FIG. 4A shows expression of CD71 on Tconvs cultured with or without dissociated islet cells from an HLA-A2-negative donor. FIG. 4B shows expression of ICOS on Tconvs cultured with or without dissociated islet cells from an HLA-A2-negative donor. FIG. 4C shows expression of CD25 on Tconvs cultured with or without dissociated islet cells from an HLA-A2-negative donor. FIG. 4D shows expression of CD71 on Tconvs cultured with or without dissociated islet cells from an HLA-A2-positive donor. FIG. 4E shows expression of ICOS on Tconvs cultured with or without dissociated islet cells from an HLA-A2-positive donor.

FIG. 4F shows expression of CD25 on Tconvs cultured with or without dissociated islet cells from an HLA-A2-positive donor.

FIGS. 5A-5C show activation of CD4+ Tregs by human islets in vitro. Tregs were cultured with or without dissociated islet cells from an HLA-A2-positive donor, and expression of the activation markers CD71, ICOS, and CD25 was measured using flow cytometry. FIG. 5A shows expression of CD71 on Tregs cultured with or without dissociated islet cells. FIG. 5B shows expression of ICOS on Tregs cultured with or without dissociated islet cells. FIG. 5C shows expression of CD25 on Tregs cultured with or without dissociated islet cells.

FIG. 6 shows glycemia in immunodeficient mice who had received a transplant of 3,000 IEQ islets from an HLA-A2-negative donor on day 3 and were subsequently injected with 8×10⁵ effector T cells (Teffs) on day 60.

FIG. 7 shows glycemia in immunodeficient mice who had received a transplant of 3,000 IEQ islets from an HLA-A2-positive donor on day 3 and were subsequently injected with 1-2×10⁶ Tregs on day 40. All mice maintained normoglycemia until the islet grafts were removed on day 73 by nephrectomizing the kidney harboring the islet graft.

FIG. 8 is a flow chart showing steps involved in production and administration of anti-DPP6 CAR expressing Tregs. In Step 4, the anti-DPP6 CAR expressing Tregs could be co-administrated with human islets or stem-cell derived beta cells when used in a transplantation setting or administered alone when used as an immunotherapy for the treatment of an autoimmune disease.

FIG. 9 shows nanobody binding to stem cell derived beta cells (SCB) as determined using flow cytometry.

FIG. 10A shows schematic diagrams of an anti-DPP6 CAR with a Myc tag inserted at the C-terminal end of the nanobody domain (anti-DPP6-cMyc CAR) or at the N-terminal end of the nanobody domain (anti-DPP6-nMyc CAR). FIG. 10B shows the transduction efficiency (mCherry+) and membrane expression (MFI Tag) of the anti-DPP6-cMyc CAR construct. FIG. 10C shows the transduction efficiency (mCherry+) and membrane expression (MFI Tag) of the anti-DPP6-nMyc CAR construct.

### DETAILED DESCRIPTION

The present disclosure relates generally to regulatory T cells (Tregs) engineered to express a dipeptidyl aminopeptidase-like protein 6 (DPP6)-reactive chimeric antigen receptor (CAR). The engineered Tregs are suitable for use in immunotherapy regimens for autoimmune, inflammatory and degenerative diseases. In particular, the anti-DPP6 CAR expressing Tregs are suitable for treating or preventing autoimmune diseases of the pancreas or central nervous system.

Tregs are a small subpopulation of peripheral blood lymphocytes and are critical for controlling tolerance, inflammation, and homeostasis of the immune system. Defects in Tregs have been observed in connection with uncontrolled inflammation and a variety of autoimmune diseases. Accordingly, Tregs are being developed as adoptive cell therapies for treating autoimmune and inflammatory diseases, graft-versus-host disease after bone marrow transplantation, and rejection of solid organ transplants (Bluestone and Tang, Science, 362:154-155, 2018).

In many autoimmune diseases, although Tregs are present, quantitative and/or qualitative defects result in an imbalance with disease-causing autoreactive effector T cells (Tconvs). Thus, restoring the balance between pathogenic Tconvs and Tregs could be a curative solution for many autoimmune diseases. Indeed, preclinical studies show therapeutic benefit of Treg infusion in a variety of autoimmune and inflammatory diseases. Importantly, for organ-specific autoimmune diseases and inflammation such as type I diabetes (T1D) and multiple sclerosis, Tregs with antigen specificity for the affected organ are often orders of magnitude more effective in halting disease. However, tissue-specific Tregs are often retained in the tissue and its draining lymph nodes, thus their frequency in the blood is very low making it difficult to isolate and expand Tregs for therapeutic use.

The past few years have witnessed exciting breakthroughs in cancer immunotherapy using T cells expressing a chimeric antigen receptor (CAR) targeting CD19. The feasibility of this approach to redirect polyclonal Tregs to a myriad of tissue antigens in pre-clinical models has also been reported. However, application of CAR technology to redirect Tregs to islet or brain antigens have not been described.

To select a CAR target for directing Tregs to pancreatic islets, proteins that are preferentially expressed in the islets were considered. Insulin is highly specific for pancreatic islets, but it is a soluble secreted protein. Although, multimeric soluble proteins can activate CARs and crystalized insulin stored inside granules may be able to trigger CARs, insulin crystals are rapidly solubilized and secreted crystalized insulin an unsuitable CAR target. Similarly, other islet hormones, such as glucagon, were also deemed to be unsuitable.

Cell surface proteins that are reported to be highly selective for pancreatic islets were considered to be viable CAR targets. Tetrapanin-7 (TSPAN7), calcium sensing receptor (CASR), prostaglandin D2 receptor 2 (PTGDR2), and dipeptidyl aminopeptidase-like protein 6 (DPP6) were selected for further assessment. Among these, CASR and PTGDR2 have multiple transmembrane and extracellular domains, and hence very complex structures, making it technically difficult to express these molecules as soluble proteins identification of binders in solution.

TSPAN-7 has three extracellular domains, one of which is large and readily expressed as a soluble protein. Initial screens in a Fab library identified several TSPAN7 binders. But unexpectedly, TSPAN-7 was found to be expressed on human B and T lymphocytes, eliminating this target from further consideration.

DPP6 is a single transmembrane protein with a larger extracellular domain. An initial screen yielded one Fab clone to DPP6. Around this time, the development of nanobodies targeting DPP6 for intravital imaging of beta cell mass was reported (Balhuizen et al., Scientific Reports, 7(1):15130, 2017). Four of the twelve anti-DPP6 (aDPP6) nanobodies, namely 2hD1, 2hD123-A24V, 2hD6 and 4hD29, were selected for CAR development based on their potential cross-reactivity with mouse DPP6 and levels of affinity.

### I. Anti-DPP6 CAR Tregs

Certain aspects of the present disclosure relate to CD4+, CD25+, CD127−/lo human regulatory T cells (Tregs) engineered to express a dipeptidyl aminopeptidase-like protein 6 (DPP6)-reactive chimeric antigen receptor (CAR) comprising an extracellular DPP6-binding domain linked through a hinge and a transmembrane domain to an intracellular domain comprising a costimulatory domain and an activation domain.

Dipeptidyl aminopeptidase-like protein 6, or “DPP6,” is a single-pass type II transmembrane protein that is also referred to as DPPX, VF2, MRD33, DPL1, dipeptidyl peptidase-like protein 6, or dipeptidyl peptidase IV-related protein. While DPP6 is a member of the peptidase S9B family of serine proteases, it does not display detectable protease activity. DPP6 is highly expressed in the human and mouse brain, and has been shown to bind specific voltage-gated potassium channels and alter their expression and biophysical properties. Variations in the DPP6 gene are associated with susceptibility to amyotrophic lateral sclerosis and with idiopathic ventricular fibrillation (Online Mendelian Inheritance in Man entry 126141; Ding et al., QJM, 111(6):373-37, 2018; and Brambilla et al., Neurosci Lett, 530(2):155-60, 2012). DPP6 has also been identified as a biomarker of endocrine cell mass that is detectable in the human pancreas (Balhuizen et al., Scientific Reports, 7(1):15130, 2017).

The DPP6 mRNA is subject to alternative splicing. In humans, there are eleven splice variants and eight protein isoforms of DPP6. The longest isoform of DPP6 is DPP6 isoform 1 (also referred to as DPP6 “L”). DPP6 isoform 1 is the DPP6 variant with the highest levels of expression in pancreatic islets (Balhuizen et al., Scientific Reports, 7(1):15130, 2017). The amino acid sequence of human DPP6 isoform 1 according to NCBI Reference Sequence NP_570629.2 is:

(SEQ ID NO: 31)
MASLYQRFTGKINTSRSFPAPPEASHLLGGQGPEEDGGAGAKPLGP
RAQAAAPRERGGGGGGAGGRPRFQYQARSDGDEEDELVGSNPPOR
NWKGIAIALLVILVICSLIVTSVILLTPAEDNSLSQKKKVTVEDL
FSEDFKIHDPEAKWISDTEFIYREQKGTVRLWNVETNTSTVLIEG
KKIESLRAIRYEISPDREYALFSYNVEPIYQHSYTGYYVLSKIPH
GDPQSLDPPEVSNAKLQYAGWGPKGQQLIFIFENNIYYCAHVGKQ
AIRVVSTGKEGVIYNGLSDWLYEEEILKTHIAHWWSPDGTRLAYA
AINDSRVPIMELPTYTGSIYPTVKPYHYPKAGSENPSISLHVIGL
NGPTHDLEMMPPDDPRMREYYITMVKWATSTKVAVTWLNRAQNVS
ILTLCDATTGVCTKKHEDESEAWLHRQNEEPVFSKDGRKFFFIRA
IPQGGRGKFYHITVSSSQPNSSNDNIQSITSGDWDVTKILAYDEK
GNKIYFLSTEDLPRRRQLYSANTVGNFNRQCLSCDLVENCTYFSA
SFSHSMDFFLLKCEGPGVPMVTVHNTTDKKKMFDLETNEHVKKAI
NDROMPKVEYRDIEIDDYNLPMQILKPATFTDTTHYPLLLVVDGT
PGSQSVAEKFEVSWETVMVSSHGAVVVKCDGRGSGFQGTKLLHEV
RRRLGLLEEKDQMEAVRTMLKEQYIDRTRVAVFGKDYGGYLSTYI
LPAKGENQGQTFTCGSALSPITDFKLYASAFSERYLGLHGLDNRA
YEMTKVAHRVSALEEQQFLIIHPTADEKIHFQHTAELITQLIRGK
ANYSLQIYPDESHYFTSSSLKQHLYRSIINFFVECFRIQDKLLTV
TAKEDEEED.
ED.

Isoform 1 is the dominant form of DPP6 expressed in pancreatic islets and the brain. The extracellular domain of isoform 1 includes residues 118-865 of SEQ ID NO:31. Isoforms 1, 2, 3 and 6 have identical extracellular domain, while isoform 4 has a small membrane-proximal truncation relative to Isoforms 1, 2, 3 and 6. In contrast, Isoform 5, 7, and 8 have very short extracellular domains. Additional information on DPP6 splice variants and protein isoforms, including nucleotide and amino acid sequence information, may be found in the NCBI Gene database, under Gene ID 1804.

The nanobodies described in Example 1 were made using a recombinant protein derived from the extracellular domain of isoform 1 as an immunogen. Thus, the nanobodies of Example 1 are expected to bind to isoforms 1, 2, 3 and 6, and possibly isoform 4, but not isoforms 5, 7 and 8. Likewise, the DPP6-binding domain of the DPP6-reactive CARs (aDPP6-CARs) of the Tregs of the present disclosure bind to the extracellular domain of isoform 1 (residues 118-865 of SEQ ID NO:31). In some embodiments, the DPP6-binding domain comprises a variable region of a DPP6-reactive nanobody. In other embodiments, the DPP6-binding domain comprises a DPP6-reactive scFv, or a DP66-reactive Fab. In some embodiments, the variable region of a DPP6-reactive nanobody comprises three complementarity-determining regions (CDRs) having amino acid sequences selected from: (i) a CDR1 of SEQ ID NO:13, a CDR2 of SEQ ID NO:14, and a CDR3 of SEQ ID NO:15; (ii) a CDR1 of SEQ ID NO:16, a CDR2 of SEQ ID NO:17, and a CDR3 of SEQ ID NO:18; (iii) a CDR1 of SEQ ID NO:19, a CDR2 of SEQ ID NO:20, and a CDR3 of SEQ ID NO:21; and (iv) a CDR1 of SEQ ID NO:22, a CDR2 of SEQ ID NO:23, and a CDR3 of SEQ ID NO:24. In some embodiments, the variable region comprises the amino acid sequence of SEQ ID NO:9, SEQ ID NO:10, SEQ ID NO:11, or SEQ ID NO:12, or an amino acid sequence sharing at least 90%, 95% or 99% identity with SEQ ID NO:9, SEQ ID NO:10, SEQ ID NO:11, or SEQ ID NO:12. In some embodiments, the variable region of the DPP6-reactive nanobody comprises one or more conservative amino acid substitution(s). In some embodiments, the conservative amino acid substitution(s) are located in a framework region of the DPP6-reactive nanobody. In other embodiments, the conservative amino acid substitution(s) are located in a CDR of the DPP6-reactive nanobody. In further embodiments, the conservative amino acid substitution(s) are located in both a framework region and a CDR of the DPP6-reactive nanobody.

The hinge of the aDPP6-CARs of the Tregs of the present disclosure connects the DPP6-binding domain to the transmembrane domain. In some embodiments, the hinge comprises an IgG4 hinge. In some embodiments, the hinge further comprises the CH3 domains of IgG4, or both the CH2 and CH3 domains of IgG4. In circumstances in which the hinge comprises the CH2 domain of IgG4, the CH2 domain may comprise one or both of L235E and N297Q substitutions. In other embodiments, the hinge comprises a CD28 hinge, or a CD8a hinge.

In exemplary embodiments, the transmembrane domain of the aDPP6-CARs of the Tregs of the present disclosure is a CD28 transmembrane domain. In other embodiments, the transmembrane domain is a CD8a transmembrane domain.

The intracellular domain of the aDPP6-CARs of the Tregs of the present disclosure comprises a costimulatory domain and an activation domain. In some embodiments, the costimulatory domain comprises a CD28 costimulatory domain. In some embodiments, the activation domain comprises a CD3 activation domain. In some embodiments, the CD3 activation domain comprises a CD3 zeta activation domain. In other embodiments, the CD3 activation domain comprises a CD3 epsilon activation domain, a CD3 delta activation domain or a CD3 gamma activation domain.

### II. Methods of Use Anti-DPP6 CAR Tregs

The anti-DPP6 CAR Tregs of the present disclosure are suitable for use in methods of treating or preventing a pathological immune response in a human subject in need thereof. In some embodiments, the pathological immune response presents as an autoimmune disease, such as an autoimmune disease of the pancreas or central nervous system. In other embodiments, the pathological immune response presents as a neurodegenerative disease. References and claims to methods comprising administering an effective amount of anti-DPP6 CAR Tregs or a pharmaceutical composition thereof to a human subject, in their general and specific forms likewise relate to:
a) the use of the anti-DPP6 CAR Tregs for the manufacture of a medicament for the treatment or prevention of a pathological immune response; and
b) pharmaceutical compositions comprising the anti-DPP6 CAR Tregs for the treatment or prevention of a pathological immune response.
   Thus, the present disclosure provides anti-DPP6 CAR Tregs for use as a medicament, for use in manufacture of a medicament, and for use in treating or preventing a pathological immune response (e.g., autoimmune disease, neurodegenerative disease, autoinflammatory disorder, etc.).

In some embodiments, the effective amount of anti-DPP6 CAR Tregs or the pharmaceutical composition comprises from 10⁵ to 10¹¹ of the human Tregs. That is, an effective amount comprises greater than or equal to 10⁵, 10⁶, 10⁷, 10⁸, 10⁹, or 10¹⁰ Tregs, and less than or equal to 10¹¹, 10¹⁰, 10⁹, 10⁸, 10⁷, or 10⁶ Tregs. In some embodiments, the effective amount of anti-DPP6 CAR Tregs or the pharmaceutical composition is administered to the human subject by intravenous infusion over an interval of from 1 to 120 minutes. That is, an effective amount is infused intravenously in an interval greater than or equal to 1, 2, 3, 5, 10, 15, 20, 25, 30, 45, 60, 75, 90 or 105 minutes, and less than or equal to 120, 05, 90, 75, 60, 45, 30, 25, 20, 15, 10, 5, 4, 3 or 2 minutes. In other embodiments, the effective amount of anti-DPP6 CAR Tregs or the pharmaceutical composition is administered to the human subject locally in conjunction with pancreatic islet or beta cell replacement therapy.

### III. Methods of Manufacture of Anti-DPP6 CAR Tregs

Certain aspects of the present disclosure relate to methods for the production of recombinant human regulatory T cells (Tregs) engineered to express a dipeptidyl aminopeptidase-like protein 6 (DPP6)-reactive chimeric antigen receptor (CAR), comprising:
a) culturing CD4+, CD25+, CD127−/lo human Tregs in medium comprising an activation agent under conditions effective in producing stimulated Tregs;
b) introducing a nucleic acid encoding a DPP6-reactive CAR comprising an extracellular DPP6-binding domain linked through a hinge and a transmembrane domain to an intracellular domain comprising a costimulatory domain and an activation domain into the stimulated Tregs under conditions effective in producing recombinant Tregs;
c) culturing the recombinant Tregs in medium comprising IL-2 under conditions effective in expanding an expanded population of recombinant Tregs; and
d) harvesting the expanded population of recombinant Tregs.

In some embodiments, the nucleic acid encoding the DPP6-reactive CAR is introduced into the human Tregs by transfection. In other embodiments, the nucleic acid encoding the DPP6-reactive CAR is introduced into the human Tregs using lentiviral transduction. In some embodiments, the nucleic acid encoding the DPP6-reactive CAR is introduced into the human Tregs using a CRISPR engineering system.

### IV Definitions

As used herein and in the appended claims, the singular form “a,” “an” and “the” includes plural forms unless indicated otherwise. For instance, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 200 fold refers to 180 fold to 220 fold and includes 200 fold).

As used herein, numerical ranges are inclusive of the numbers defining the range (e.g., 10 to 20 amino acids encompasses 10, 11, 12, 13, 14, 15, 16, 17, 18, 19 and 20 amino acids.

Exemplary amino acid sequences are set forth in sequence identifiers throughout the present disclosure. Some of the claimed embodiments are described by reference to a percent identity shared with an exemplary amino acid sequence. Two amino acid sequences are substantially identical if their amino acid sequences share at least 90% identity (e.g., at least 90%, 91%, 92%, 93%, 94%, 95%, 96%, 97%, 98%, 99% or 100% identity over a specified region, or, when not specified, over their entire sequences), when compared and aligned for maximum correspondence over a comparison window or designated region. As pertains to the present disclosure and claims, the BLASTP sequence comparison algorithm using default parameters is used to align amino acid sequences for determination of sequence identity.

Algorithms that are suitable for determining percent sequence identity and sequence similarity are the BLAST and BLAST 2.0 algorithms, described in Altschul et al., J Mol Biol, 215: 403-410, 1990; and Altschul et al., Nucleic Acids Res. 25: 3389-3402, 1977, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff and Henikoff, Proc. Natl. Acad. Sci. USA 89:10915, 1989).

As used herein, the term “isolated” refers to an object (e.g., Tregs) that is removed from its environment (e.g., separated). “Isolated” objects are at least 50% free, preferably 75% free, more preferably at least 90% free, and most preferably at least 95% (e.g., 95%, 96%, 97%, 98%, or 99%) free from other components with which they are associated.

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., human Tregs) effective to “treat” a disease or disorder in a subject (e.g., a mammal such as a human). An “effective amount” or an “amount sufficient” of an agent may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission. “Treatment” can also mean prolonging survival of a recipient of an allograft as compared to expected survival of a recipient of an allograft not receiving treatment. “Palliating” a disease or disorder means that the extent and/or undesirable clinical manifestations of the disease or disorder are lessened and/or time course of progression of the disease or disorder is slowed, as compared to the expected untreated outcome.

As used herein, the term “pathological immune response” encompasses autoimmune diseases, and autoinflammatory diseases. “Autoimmune diseases” involve immune recognition resulting in direct damage to self-tissue and functional impairments. Pathologically, autoimmune diseases are typically driven by cells of the adaptive immune system. An example of an autoimmune disease is type I diabetes. “Autoinflammatory diseases” involve spontaneous activation, or over-reaction of the immune system to non-self-antigens (e.g., environmental, food, commensal or other antigens) resulting in indirect (bystander) damage to self-tissue and functional impairments. Pathologically, autoinflammatory diseases are typically dominated by cells of the innate immune system. An example of an autoinflammatory disease is the neurodegenerative disease known as Lou Gehrig's disease or amyotrophic lateral sclerosis.

### ENUMERATED EMBODIMENTS

1. A human regulatory T cell (Treg) engineered to express a dipeptidyl aminopeptidase-like protein 6 (DPP6)-reactive chimeric antigen receptor (CAR), wherein the Treg is CD4+, CD25+, CD127−/lo, and the CAR comprises an extracellular DPP6-binding domain linked through a hinge and a transmembrane domain to an intracellular domain comprising a costimulatory domain and an activation domain.
2. The human Treg of embodiment 1, wherein the extracellular DPP6 binding domain comprises a variable region of a DPP6-reactive nanobody, wherein the variable region comprises three complementarity-determining regions (CDRs) having amino acid sequences selected from:
(i) a CDR1 of SEQ ID NO:13, a CDR2 of SEQ ID NO:14, and a CDR3 of SEQ ID NO:15;
(ii) a CDR1 of SEQ ID NO:16, a CDR2 of SEQ ID NO:17, and a CDR3 of SEQ ID NO:18;
(iii) a CDR1 of SEQ ID NO:19, a CDR2 of SEQ ID NO:20, and a CDR3 of SEQ ID NO:21; and
(iv) a CDR1 of SEQ ID NO:22, a CDR2 of SEQ ID NO:23, and a CDR3 of SEQ ID NO:24.
3. The human Treg of embodiment 1 or embodiment 2, wherein the variable region comprises an amino acid sequence sharing at least 90%, 95% or 99% identity with SEQ ID NO:9, SEQ ID NO:10, SEQ ID NO:11, or SEQ ID NO:12.
4. The human Treg of any one of embodiments 1-3, wherein the hinge is an IgG4 hinge.
5. The human Treg of embodiment 4, wherein the hinge comprises an amino acid sequence sharing at least 90% identity with SEQ ID NO:3.
6. The human Treg of any one of embodiments 1-5, wherein the transmembrane domain is a CD28 transmembrane domain.
7. The human Treg of embodiment 6, wherein the transmembrane domain comprises an amino acid sequence sharing at least 90%, 95% or 99% identity with SEQ ID NO:4.
8. The human Treg of any one of embodiments 1-7, wherein the costimulatory domain comprises a CD28 costimulatory domain.
9. The human Treg of embodiment 8, wherein the CD28 costimulatory domain comprises an amino acid sequence sharing at least 90%, 95% or 99% identity with SEQ ID NO:5.
10. The human Treg of any one of embodiments 1-9, wherein the activation domain comprises a CD3 zeta activation domain.
11. The human Treg of embodiment 10, wherein the CD3 zeta activation domain comprises an amino acid sequence sharing at least 90%, 95% or 99% identity with SEQ ID NO:6.
12. The human Treg of any one of embodiments 1-11, wherein the hinge, the transmembrane domain and the intracellular domain comprise an amino acid sequence sharing at least 90%, 95% or 99% identity with SEQ ID NO:25.
13. The human Treg of any one of embodiments 1-12, wherein the DPP6-reactive CAR further comprises an N-terminal signal peptide.
14. The human Treg of embodiment 13, wherein the N-terminal signal peptide comprises an amino acid sequence sharing at least 90%, 95% or 99% identity with SEQ ID NO:1.
15. The human Treg of any one of embodiments 1-14, further comprising a tag, optionally wherein the tag is a myc tag of SEQ ID NO:2 or a strep tag of SEQ ID NO:26, optionally wherein the tag is located on the N-terminal or the C-terminal side of the DPP6-binding domain.
16. The human Treg of any one of embodiments 1-15, wherein the intracellular domain further comprises a self-cleaving peptide and a label C-terminal to the activation domain.
17. The human Treg of embodiment 16, wherein the self-cleaving peptide is P2A, optionally wherein the self-cleaving peptide comprises an amino acid sequence sharing at least 90%, 95% or 99% identity with SEQ ID NO:7.
18. The human Treg of any one of embodiments 1-17, wherein the label is a fluorescent protein, optionally wherein the fluorescent protein is mCherry, optionally wherein mCherry comprises an amino acid sequence sharing at least 90%, 95% or 99% identity with SEQ ID NO:8.
19. The human Treg of any one of embodiments 1-15, wherein the DPP6-reactive CAR comprises an amino acid sequence sharing at least 90%, 95% or 99% identity with SEQ ID NO:27, SEQ ID NO:28, SEQ ID NO:29, or SEQ ID NO:30.
20. The human Treg of any one of embodiments 1-19, wherein the human Treg is FOXP3+, HELIOS+.
21. The human Treg of any one of embodiments 1-20, wherein the human Treg has a FoxP3 promoter with a demethylated Treg-specific demethylation region (TSDR).
22. The human Treg of any one of embodiments 1-21, for use in treating or preventing type I diabetes in a human subject in need thereof.
23. The human Treg of any one of embodiments 1-21, for use in reducing hyperglycemia in a human subject in need thereof.
24. The human Treg of any one of embodiments 1-21, for use in preventing death of pancreatic beta-cells in a human subject in need thereof.
25. The human Treg of any one of embodiments 1-21, for use in treating or preventing an autoimmune disease of the nervous system in a human subject in need thereof, optionally for treating or preventing autoimmune encephalitis or multiple sclerosis.
26. The human Treg of any one of embodiments 1-21, for use in treating or preventing a neurodegenerative disease in a human subject in need thereof, optionally for treating or prevent a disease selected from frontotemporal dementia, amyotrophic lateral sclerosis, progressive supranuclear palsy, Alzheimer's disease, and Parkinson's disease.
27. A method for the production of recombinant human regulatory T cells (Tregs) engineered to express a dipeptidyl aminopeptidase-like protein 6 (DPP6)-reactive chimeric antigen receptor (CAR), the method comprising:
a) culturing CD4+, CD25+, CD127−/lo human Tregs in medium comprising an activation agent under conditions effective in producing stimulated Tregs;
b) introducing a nucleic acid encoding a DPP6-reactive CAR comprising an extracellular DPP6-binding domain linked through a hinge and a transmembrane domain to an intracellular domain comprising a costimulatory domain and an activation domain into the stimulated Tregs under conditions effective in producing recombinant Tregs;
c) culturing the recombinant Tregs in medium comprising IL-2 under conditions effective in expanding an expanded population of recombinant Tregs; and
d) harvesting the expanded population of recombinant Tregs.
28. The method of embodiment 27, wherein step c) further comprises culturing the expanded population of recombinant Tregs in medium comprising an activation agent under conditions effective in producing restimulated Tregs.
29. The method of embodiment 27 or embodiment 28, wherein the activation agent comprises CD3 and CD28 agonists for cross-linking CD3 and CD28 of the Tregs, optionally wherein the CD3 and CD28 agonists comprise monoclonal antibodies or fragments thereof coupled to a multimerization agent, optionally wherein the multimerization agent comprises a superparamagnetic bead or a polymeric matrix, optionally wherein the multimerization agent comprises Fc receptor-expressing feeder cells.
30. The method of embodiment 27 or embodiment 28, wherein the activation agent comprises a CD28 superagonist antibody in the absence of an anti-CD3 antibody.
31. The method of any one of embodiments 27-30, wherein the CD4+, CD25+, CD127−/low T cells of step a) are isolated by fluorescence-activated cell sorting (FACS) or magnetic-activated cell sorting (MACS) from a lymphocyte-containing biological sample obtained from a human subject.
32. The method of embodiment 31, wherein the lymphocyte-containing biological sample is selected from the group consisting of whole blood, a leukapheresis product, and peripheral blood mononuclear cells.
33. The method of embodiment 31 or embodiment 32, wherein the lymphocyte-containing biological sample is either fresh or cryopreserved and thawed after being obtained from the human subject.
34. A pharmaceutical composition comprising from 10⁷ to 10¹¹ of the human Tregs of any one of embodiments 121, or produced by the methods of any one of embodiments 27-32, and a physiologically acceptable buffer.
35. A method of treating or preventing type I diabetes in a human subject in need thereof, wherein the method comprises administering a therapeutically effective amount of the pharmaceutical composition of embodiment 34.

### EXAMPLES

The present disclosure is described in further detail in the following examples, which are not intended to limit the scope of the disclosure as claimed. The attached figures are meant to be considered as integral parts of the specification and description of the disclosure. The following examples are offered to illustrate, but not to limit the claimed disclosure.

In the experimental disclosure which follows, the following abbreviations apply: Ab (antibody); CAR (chimeric antigen receptor); DPP6 (dipeptidyl aminopeptidase-like protein 6); FACS (fluorescence-activated cell sorting); HLA (human leukocyte antigen); IEQ (islet equivalent); IL-2 (interleukin-2); MFI (mean fluorescent intensity); MOI (multiplicity of infection); NSG (NOD SCID Gamma); PBMC (peripheral blood mononuclear cell); SBC (stem cell-derived beta cells); STII (Streptavidin Tag II); STZ (streptozotocin); Tconv (conventional T cell); Teff (effector T cell); Treg (regulatory T cell); TSDR (Treg-specific demethylation region); and UCSF (University of California San Francisco).

### Example 1

### Anti-Dipeptidyl Aminopeptidase-Like Protein 6 (DPP6)-Reactive Chimeric Antigen Receptor (CAR)-Bearing T Cells

This example describes the generation of anti-DPP6 CAR constructs, as well as human T cells engineered to express the anti-DPP6 CARs (aDPP6 CAR T cells).

Materials and Methods

Generation of anti-DPP6 CAR constructs. The anti-DPP6 nanobodies 2hD1, 2hD123-A24V, 2hD6 and 4hD29 were used to generate anti-DPP6 CAR constructs. DNA sequences corresponding to each nanobody followed by a tag (e.g., C-terminal myc tag) were synthesized. Each DNA sequence was further ligated following the Gibson assembly protocol to a pre-digested in-lab vector containing an IgG4 hinge, CD28 transmembrane and intracellular domains, a CD3zeta intracellular domain, a P2A self-cleaving peptide and mCherry DNA sequences (Table 1-1), as well as the ampicillin resistance gene. Competent E. coli were transformed with the different ligation products. Five colonies/plate were picked and overnight cultures were grown for DNA purification. Successful transformation and ligation was confirmed by performing DNA sequencing of the ligation products. The CAR constructs were further cloned into a lentiviral vector with generation 2 backbone. The amino acid sequences of the CAR domains and anti-DPP6 nanobodies are provided in Table 1-1 and Table 1-2. In Table 1-3, the amino acid sequence shared by the four CARs is set forth as SEQ ID NO:25 (IgG4 Hinge+CD28 TM+CD28 endo+CD3z), while the amino acid sequences of the mature CARs (absent signal peptide, P2A and mCherry) are set forth as SEQ ID NOs:27-30.

TABLE 1-1
Amino Acid Sequences of Chimeric
Antigen Receptor (CAR) Domains

| Domain | Amino Acid Sequence |
|---|---|
| Signal | MALPVTALLLPLALLLHAARP |
| | (SEQ ID NO: 1) |
| Nanobody | See Table 1-2 |
| Myc Tag | EQKLISEEDL (SEQ ID NO: 2) |
| IgG4 Hinge | ESKYGPPCPPCP (SEQ ID NO: 3) |
| CD28 TM | MFWVLVVVGGVLACYSLLVTVAFII |
| | FWV (SEQ ID NO: 4) |
| CD28 endo | RSKRSRLLHSDYMNMTPRRPGPTRK |
| | HYQPYAPPRDFAAYRS (SEQ ID NO: 5) |
| CD3z | RVKFSRSADAPAYQQGQNQLYNELN |
| | LGRREEYDVLDKRRGRDPEMGGKPR |
| | RKNPQEGLYNELQKDKMAEAYSEIG |
| | MKGERRRGKGHDGLYQGLSTATKDT |
| | YDALHMQALPPR (SEQ ID NO: 6) |
| P2A | ATNFSLLKQAGDVEENPGP |
| | (SEQ ID NO: 7) |
| mCherry | MVSKGEEDNMAIIKEFMRFKVHMEG |
| | SVNGHEFEIEGEGEGRPYEGTQTAK |
| | LKVTKGGPLPFAWDILSPQFMYGSK |
| | AYVKHPADIPDYLKLSFPEGFKWER |
| | VMNFEDGGVVTVTQDSSLQDGEFIY |
| | KVKLRGTNFPSDGPVMQKKTMGWEA |
| | SSERMYPEDGALKGEIKQRLKLKDG |
| | GHYDAEVKTTYKAKKPVQLPGAYNV |
| | NIKLDITSHNEDYTIVEQYERAEGR |
| | HSTGGMDELYK (SEQ ID NO: 8) |

TABLE 1-2
Amino Acid Sequences of Variable Regions
of Anti-DPP6 Nanobody Clones

| Clone | Amino Acid Sequence |
|---|---|
| | Variable Region |
| 2hD1 | QVQLQESGGGLVQPGGSLRLSCAASGYPYGYTFSS |
| | YCMRWFRQAPGKDREGVARFERNGLTTYYDDSVKG |
| | RFTISQDNVKNTVYLQMNSLKPEDTATYYCAAAPK |
| | QLRTCGDYNYWGQGTQVTVSS |
| | (SEQ ID NO: 9) |
| 2hD123_ | QVQLQESGGGSVQAGGSLRLSCAVSGSSYSRFRMG |
| A24V | WFRQVPGKEREGVAAIYRSDGRTYYADSVKGRFTI |
| | SQDNTKNTVYLQMNSLKPEDTAMYYCAAGAYSSYL |
| | MDANFAYWGQGTQVTVSS |
| | (SEQ ID NO: 10) |
| 2hD6 | QVQLQESGGGSVQAGGSLRLSCAASSYTYSYSCMA |
| | WFRQAPGKERERVASIHTGT |
| | GTANYADSAKGRFTISQDIAANTVYLQMNSLKLED |
| | TAMYYCAARPGSAALRCTTDYSKPHDFTYWGQGTQ |
| | VTVSS |
| | (SEQ ID NO: 11) |
| 4hD29 | QVQLQESGGGLVQPGGSLRLSCAASGFTFSSNYMT |
| | WVRQAPGKGPEWVSGINPDGSSTYYADSVKGRFTI |
| | SRDNAKNTLYLQMNSLKSEDTALYKCATGAAPRIP |
| | TTLRGQGTQVTVSS |
| | (SEQ ID NO: 12) |
| | Complementarity Determining Regions |
| 2hD1 | CDR1: GYPYGYTFSSYC (SEQ ID NO: 13) |
| | CDR2: FERNGLTT (SEQ ID NO: 14) |
| | CDR3: AAAPKQLRTCGDYNY (SEQ ID NO: 15) |
| 2hD123- | CDR1: GSSYSRFR (SEQ ID NO: 16) |
| A24V | |
| | CDR2: IYRSDGRT (SEQ ID NO: 17) |
| | CDR3: AAGAYSSYLMDANFAY (SEQ ID NO: 18) |
| 2hD6 | CDR1: SYTYSYSC (SEQ ID NO: 19) |
| | CDR2: IHTGTGTA (SEQ ID NO: 20) |
| | CDR3: AARPGSAALRCTTDYSKPHDFTY |
| | (SEQ ID NO: 21) |
| 4hD29 | CDR1: GFTFSSNY (SEQ ID NO: 22) |
| | CDR2: INPDGSST (SEQ ID NO: 23) |
| | CDR3: ATGAAPRIPTTL (SEQ ID NO: 24) |

TABLE 1-3
Amino Acid Sequences of Anti-DPP6 CARs\hat{ }

| | |
|---|---|
| Shared | ESKYGPPCPPCPMFWVLVVV |
| | GGVLACYSLLVTVAFIIFWV |
| | RSKRSRLLHSDYMNMTPRRP |
| | GPTRKHYQPYAPPRDFAAYR |
| | SRVKFSRSADAPAYQQGQNQ |
| | LYNELNLGRREEYDVLDKRR |
| | GRDPEMGGKPRRKNPQEGLY |
| | NELQKDKMAEAYSEIGMKGE |
| | RRRGKGHDGLYQGLSTATKD |
| | TYDALHMQALPPR |
| | (SEQ ID NO: 25) |
| Clone | |
| 2hD1 | QVQLQESGGGLVQPGGSLRL |
| | SCAASGYPYGYTFSSYCMRW |
| | FRQAPGKDREGVARFERNGL |
| | TTYYDDSVKGRFTISQDNVK |
| | NTVYLQMNSLKPEDTATYYC |
| | AAAPKQLRTCGDYNYWGQGT |
| | QVTVSS |
| | [X]n |
| | ESKYGPPCPPCPMFWVLVVV |
| | GGVLACYSLLVTVAFIIFWV |
| | RSKRSRLLHSDYMNMTPRRP |
| | GPTRKHYQPYAPPRDFAAYR |
| | SRVKFSRSADAPAYQQGQNQ |
| | LYNELNLGRREEYDVLDKRR |
| | GRDPEMGGKPRRKNPQEGLY |
| | NELQKDKMAEAYSEIGMKGE |
| | RRRGKGHDGLYQGLSTATKD |
| | TYDALHMQALPPR |
| | (SEQ ID NO: 27) |
| 2hD123-A24V | QVQLQESGGGSVQAGGSLRL |
| | SCAVSGSSYSRFRMGWFRQV |
| | PGKEREGVAAIYRSDGRTYY |
| | ADSVKGRFTISQDNTKNTVY |
| | LQMNSLKPEDTAMYYCAAGA |
| | YSSYLMDANFAYWGQGTQVT |
| | VSS |
| | [X]n |
| | ESKYGPPCPPCPMFWVLVVV |
| | GGVLACYSLLVTVAFIIFWV |
| | RSKRSRLLHSDYMNMTPRRP |
| | GPTRKHYQPYAPPRDFAAYR |
| | SRVKFSRSADAPAYQQGQNQ |
| | LYNELNLGRREEYDVLDKRR |
| | GRDPEMGGKPRRKNPQEGLY |
| | NELQKDKMAEAYSEIGMKGE |
| | RRRGKGHDGLYQGLSTATKD |
| | TYDALHMQALPPR |
| | (SEQ ID NO: 28) |
| 2hD6 | QVQLQESGGGSVQAGGSLRL |
| | SCAASSYTYSYSCMAWFRQA |
| | PGKERERVASIHTGTGTANY |
| | ADSAKGRFTISQDIAANTVY |
| | LQMNSLKLEDTAMYYCAARP |
| | GSAALRCTTDYSKPHDFTYW |
| | GQGTQVTVSS |
| | [X]n |
| | ESKYGPPCPPCPMFWVLVVV |
| | GGVLACYSLLVTVAFIIFWV |
| | RSKRSRLLHSDYMNMTPRRP |
| | GPTRKHYQPYAPPRDFAAYR |
| | SRVKFSRSADAPAYQQGQNQ |
| | LYNELNLGRREEYDVLDKRR |
| | GRDPEMGGKPRRKNPQEGLY |
| | NELQKDKMAEAYSEIGMKGE |
| | RRRGKGHDGLYQGLSTATKD |
| | TYDALHMQALPPR |
| | (SEQ ID NO: 29) |
| 4hD29 | QVQLQESGGGLVQPGGSLRL |
| | SCAASGFTFSSNYMTWVRQA |
| | PGKGPEWVSGINPDGSSTYY |
| | ADSVKGRFTISRDNAKNTLY |
| | LQMNSLKSEDTALYKCATGA |
| | APRIPTTLRGQGTQVTVSS |
| | [X]n |
| | ESKYGPPCPPCPMFWVLVVV |
| | GGVLACYSLLVTVAFIIFWV |
| | RSKRSRLLHSDYMNMTPRRP |
| | GPTRKHYQPYAPPRDFAAYR |
| | SRVKFSRSADAPAYQQGQNQ |
| | LYNELNLGRREEYDVLDKRR |
| | GRDPEMGGKPRRKNPQEGLY |
| | NELQKDKMAEAYSEIGMKGE |
| | RRRGKGHDGLYQGLSTATKD |
| | TYDALHMQALPPR |
| | (SEQ ID NO: 30) |

\hat{ }[X]n represents an optional tag of up to 20 amino acids in length. The tag if present, may be adjacent to either the N-terminus or the C-terminus (shown) of the DP66-binding domain (nanobody sequence). In [X]n, n is an integer from 0 to 20, and each X is independently selected from any amino acid or missing.

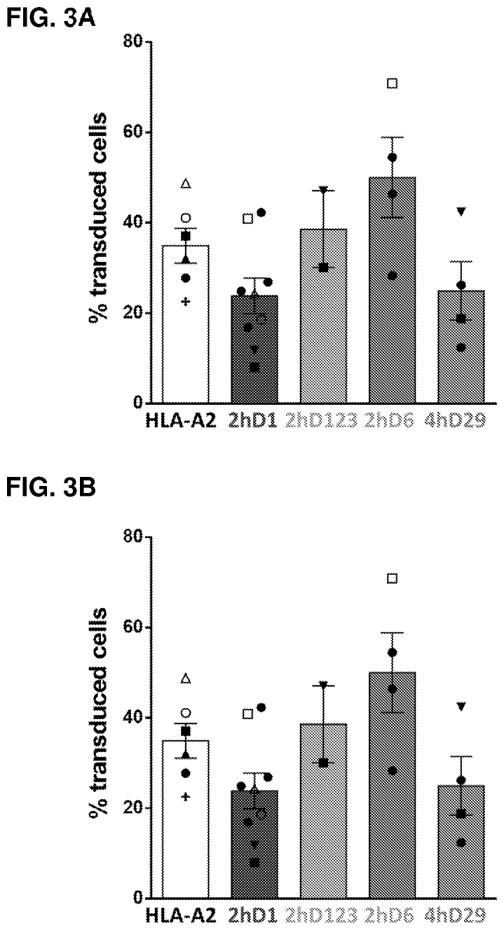

Transduction of anti-DPP6 CARs constructs into primary human T cells. Heparinized venous blood was collected from healthy donors, diluted 1:1 with PBS and then layered on a density gradient (Ficoll). Peripheral blood mononuclear cells (PBMCs) were collected from the interface after centrifugation, washed with PBS+FBS2%, and resuspended in PBS+FBS2%+EDTA2 mM. CD4+ Tconvs and Tregs were purified after negative enrichment of CD4+ cells (EasySep™ Human CD4+ T Cell Isolation Kit, StemCell), followed by CD4, CD25 and CD127 staining and cell sorting of CD4+ CD25low/−CD127hi cells (Tconvs) and CD4+ CD25hi CD127low cells (Tregs) using the BD FACSAria II. Human CD4+ Tconvs and Tregs were stimulated for 48 hours with aCD3/aCD28 Dynabeads at 1:1 ratio and cultured in complete RPMI supplemented with IL2 (100 IU/ml for Tconvs, 300 IU/ml for Tregs). Cells were next spin-fected by adding virus at an MOI of 1:1 and polybrene at 5 ug/ml final. The culture was then checked every other day and fresh complete RPMI supplemented with IL2 was added when needed. CAR transduction efficiency and membrane expression were evaluated 5 days after transduction by assessing intracellular expression of mCherry protein and membrane expression of the Tag (FIGS. 3A-3B). For Tconvs only, aCD3/aCD28 Dynabeads were removed that same day.

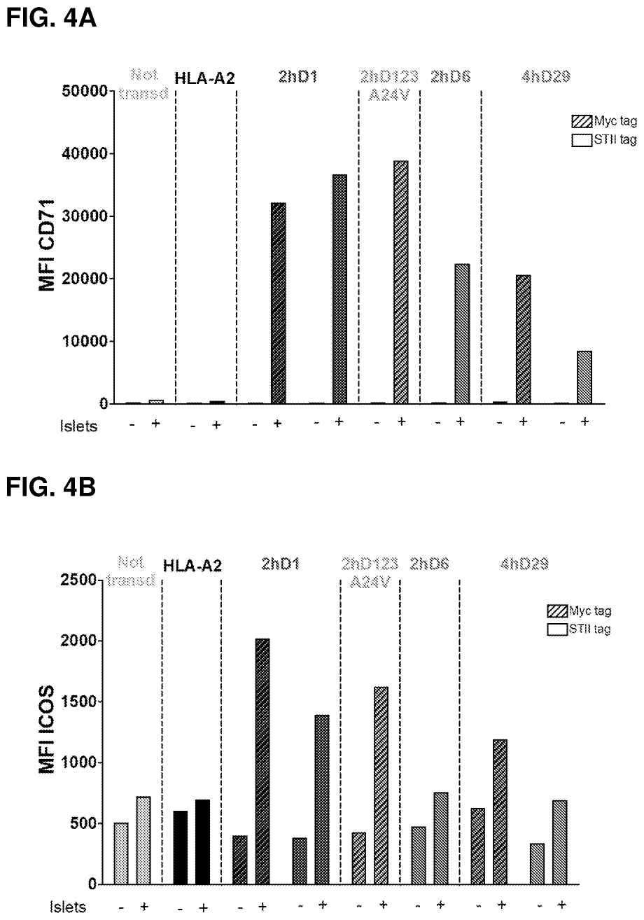

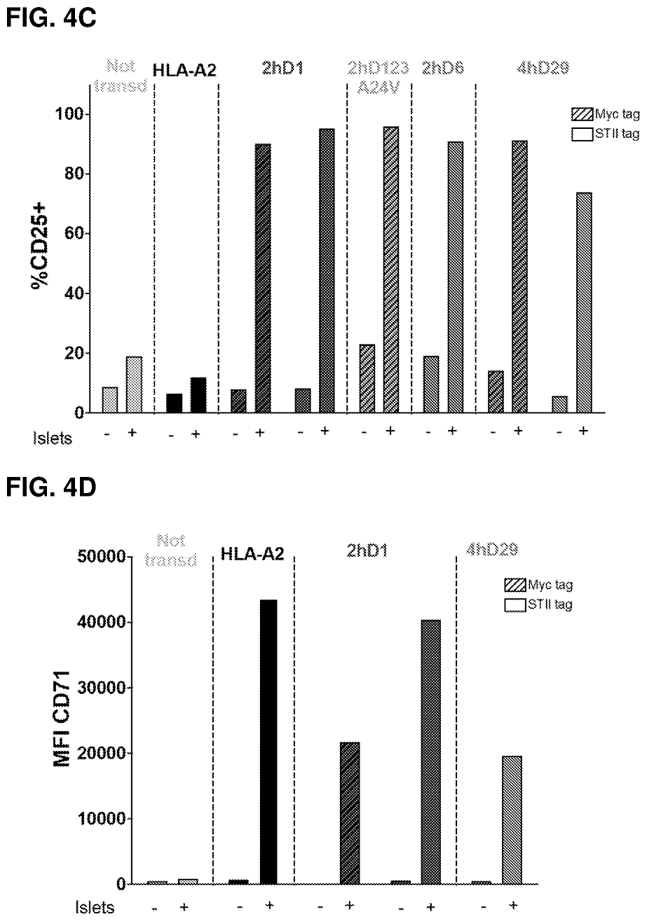

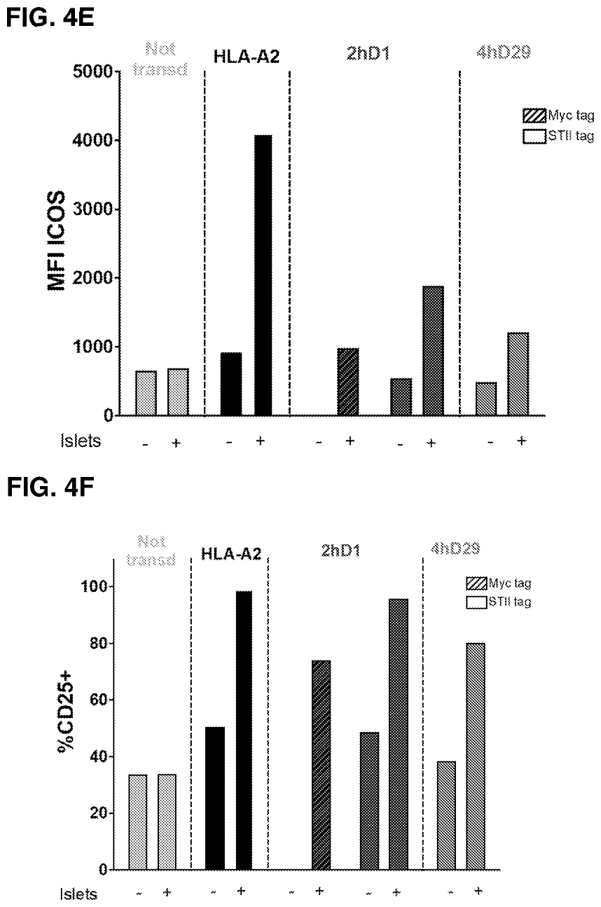

Anti-DPP6 CAR activation of CD4+ Tconvs by human islets in vitro. Pancreas from deceased non-diabetic donors was harvested and digested. Islets were hand-picked and put in MIAMI medium. Islets were then enzymatically dissociated (Accumax) and washed in MIAMI medium. After counting, islet cells were resuspended at 1×10⁶ cells/ml in MIAMI medium and 100 ul/well of the cell suspension were dispatched to wells of a 96-well round bottom plate. mCherry/CAR+ Tconvs (CD4+CD25low/−CD127hi cells) were sorted 7 days after transduction and kept in culture with complete RPMI+IL2 (100 IU/ml) for 3 more days. Cells were then used fresh, or frozen and thawed when islets were available. Tconvs were cultured for 24 hours with complete RPMI+IL2 prior to co-culture with islets. CAR+ Tconvs were resuspended at 1×10⁶ cells/ml in MIAMI medium and 100 ul/well of the cell suspension were added to wells of the 96-well round bottom plate containing the dissociated islets. Cells were co-cultured for 48 hours at 37° C. At the end of the culture, Tconv activation was assessed by staining for CD71, ICOS and CD25. Samples were acquired on a BD FortessaX20 cytometer and analyzed using the FlowJo software. Untransduced and anti-HLA-A2 CAR-transduced CD4+ Tconvs from the same donor were used as controls (FIGS. 4A-4F).

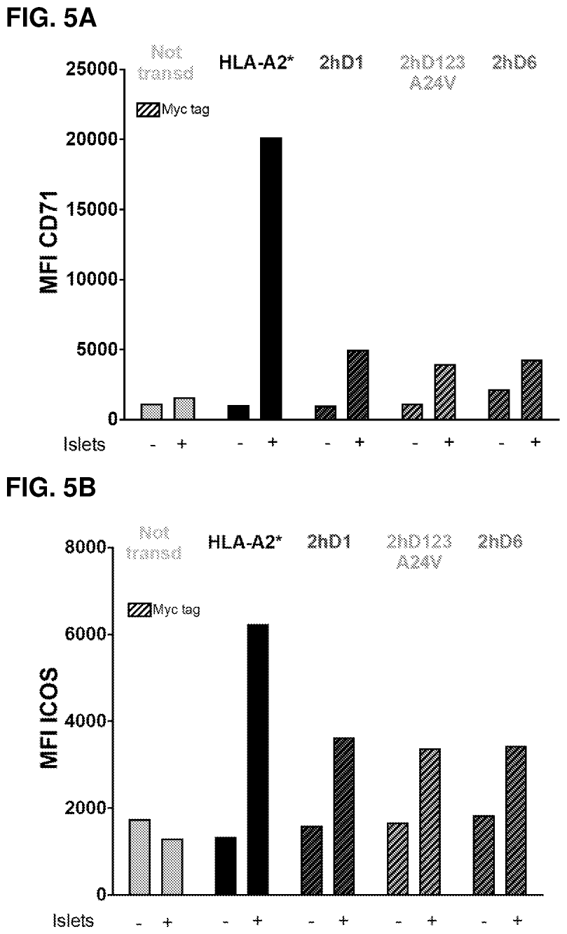

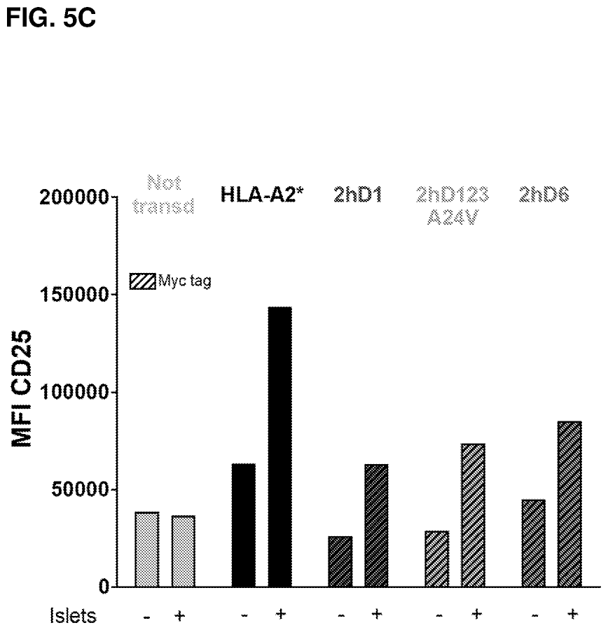

Anti-DPP6 CAR activation of CD4+ Tregs by human islets in vitro. Pancreas from deceased non-diabetic donors was harvested and digested. Islets were hand-picked and put in MIAMI medium. Islets were then enzymatically dissociated (Accumax) and washed in MIAMI medium. After counting, islet cells were resuspended at 1×10⁶ cells/ml in MIAMI medium and 100 ul/well of the cell suspension were dispatched to wells of a 96-well round bottom plate. mCherry/CAR+ Tregs (CD4+CD25hi CD127low/− cells) were sorted 7 days after transduction and kept in culture with complete RPMI+IL2 (300 IU/ml)+aCD3/aCD28 Dynabeads (1:1 ratio) for 3 more days. Cells were then used fresh, or frozen and thawed when islets were available. Tregs were cultured for 24 hours with complete RPMI+IL2, without TCR stimulation, prior to co-culture with islets. CAR+ Tregs were resuspended at 1×10⁶ cells/ml in MIAMI medium plus IL2 and 100 ul/well of the cell suspension were added to wells of the 96-well round bottom plate containing the dissociated islets. Cells were co-cultured for 48 hours at 37° C. At the end of the culture, Treg activation was assessed by staining for CD71, ICOS and CD25. Samples were acquired on a BD FortessaX20 cytometer and analyzed using the FlowJo software. Untransduced and anti-HLA-A2 CAR-transduced Tregs from the same donor were used as controls (FIGS. 5A-5C).

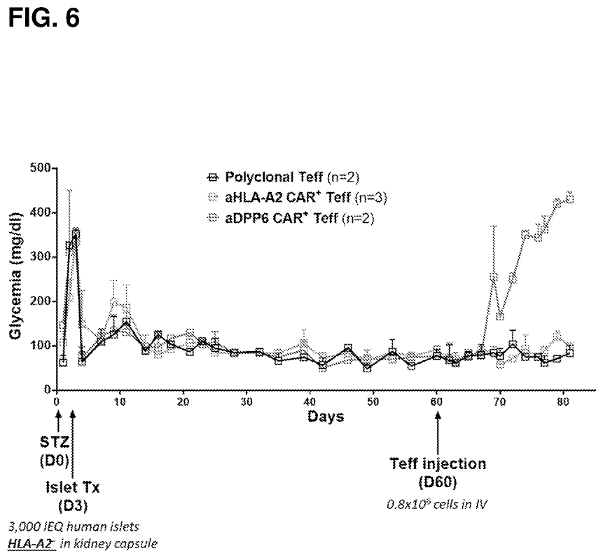

Administration of anti-DPP6 CAR expressing CD4+ Tconvs to mice. Streptozotocin (STZ) was injected into NSG mice (216 mg/kg) to deplete endogenous mouse islets. Once mice became diabetic (i.e. glycemia>300 mg/dl and ketone detected in the blood) 3,000IEQ human islets were transplanted into the kidney capsule, and glucose levels were monitored every other day. Once mouse glycemia was stably normalized, 0.8×10⁶ CAR+ or polyclonal CD4+ Tconvs were intravenously injected and glucose levels were monitored every other day (FIG. 6). Mice were sacrificed at day 80 and the spleen, islet-engrafted kidney and pancreas were harvested for immunofluorescent staining.

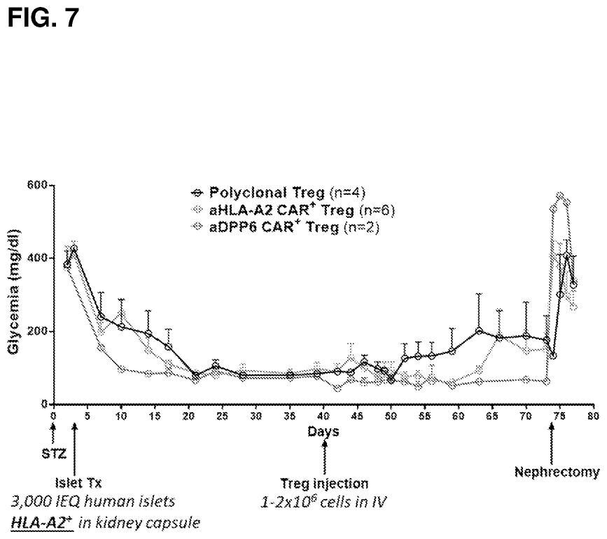

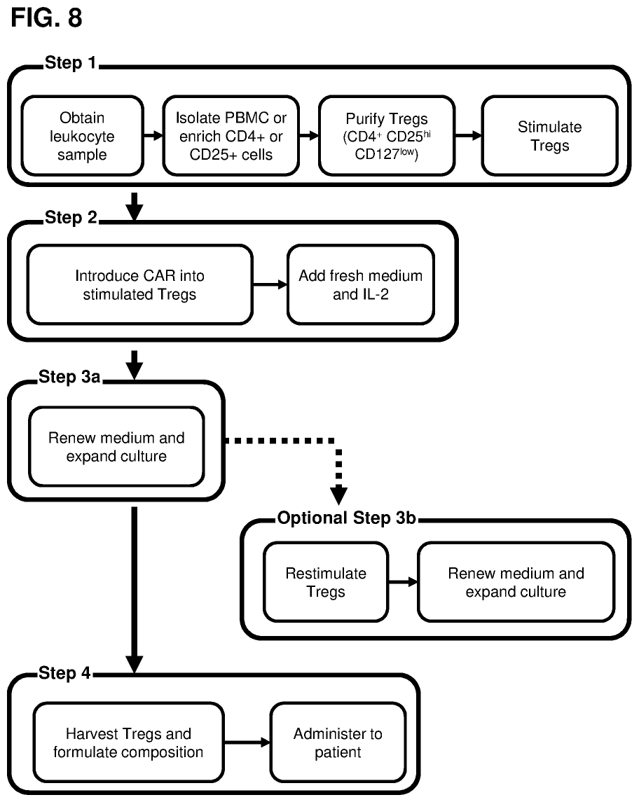

Administration of anti-DPP6 CAR expressing Tregs to mice. STZ was injected into NSG mice and 3,000IEQ human islets were transplanted into the right kidney capsule once mice were diabetic, as described above. Glucose levels were monitored every other day. Once mouse glycemia was stably normalized, 1 to 2×10⁶ CAR+ or polyclonal luciferase-expressing Tregs were intravenously injected at day 40 and 25,000U/mouse of IL2 was administered twice a day for 8 days. Glucose monitoring was performed every other day. At day 73, nephrectomy of the kidney transplanted with human islets was performed on all mice, and glucose levels were monitored every day (FIG. 7). In parallel, the in vivo migration of the cells was evaluated by bioluminescence after intraperitoneal injection of luciferin at a dose of 1 mg/mouse.

Assessment of anti-DPP6 CAR-expressing CD4+ Tconvs in vivo binding to mouse DPP6. Human CD4+ Tconvs were transduced to express luciferase and anti-HLA-A2 CAR or an anti-DPP6 CARs (2hD1 or 2hD6 clones), as previously described. Five days after transduction, aCD3/aCD28 Dynabeads were removed, transduction assessed and 2 days later anti-DPP6 CAR-expressing Tconvs were sorted. One week later, the different CD4+ Tconv cultures were counted and injected intravenously at the following doses: 2 or 4 million cells of polyclonal CD4+ Tconvs; 4 million aHLA-A2 CAR CD4+ Tconvs; 1.5 million (2hD-1) or 2.5 million (2hD-6) aDPP6 CAR CD4+ Tconvs. Bioluminescence imaging was performed 2, 4, 6 and 9 days after T cell injection. After the last bioluminescence imaging, mice were sacrificed and the spleen, lung, brain, liver, spinal cord and leg bone were harvested, put in a bath of diluted luciferin, and imaged. Organ specimens were also saved to perform further immunofluorescent stainings.

### Results

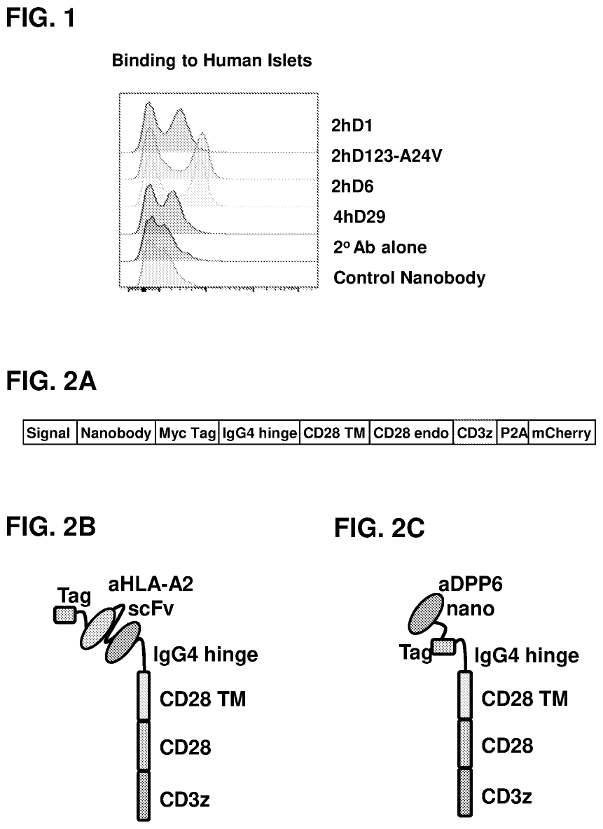

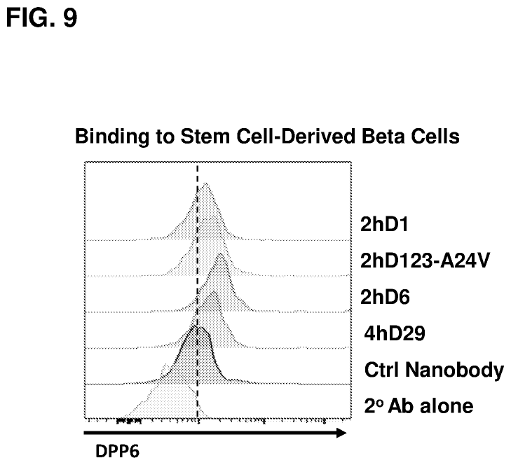

To generate anti-DPP6 CARs, the anti-DPP6 nanobodies 2hD1, 2hD123-A24V, 2hD6 and 4hD29 were selected based on their potential cross-reactivity with mouse DPP6 and their various levels of affinity (Table 1-4). Binding of the nanobodies to primary human islets was verified using flow cytometry (FIG. 1). In addition, stem cell-derived beta cells (SBC) were obtained from human embryonic stem cells according to published methods (Nair et al., Nature Cell Biology, 21:263-274, 2019; and Nair et al., Prot Exchange, 2019). Binding of the nanobodies to the SBC was also verified using flow cytometry (FIG. 9).

TABLE 1-4
Cross-Reactivity and Affinity of anti-DPP6 Nanobodies

| | CROSS-REACTIVITY | AFFINITY |
|---|---|---|
| NANOBODY | (M, H) | (K_{D}, NMOL/L) |
| 2HD-38 | | 145 |
| 2HD-1 | YES | 69 |
| 4HD-29 | | 1.2 |
| 2HD-123 | YES | 200 |
| 2HD-6 | | 13 |
| 2HD123_A24V | YES | 27.2 |

The nanobodies were used to generate anti-DPP6 CAR constructs (FIG. 2A), and the constructs were transduced into primary human T cells. An average transduction efficiency of 35% was achieved for Tconvs (FIG. 3A), and a slightly lower average transduction efficiency of 28% was achieved for Tregs (FIG. 3B). In both types of T cells, CAR membrane expression was not fully proportional to the level of cell transduction and was lower in the Tregs.

To evaluate the in vitro functionality of the anti-DPP6 CAR-expressing human T cells, the engineered T cells were co-incubated with primary dissociated human islets, and T cell activation was assessed. Specifically, anti-DPP6 CAR-expressing CD4+ Tconvs (FIGS. 4A-4F) or Tregs (FIGS. 5A-5C) were cultured with or without dissociated islet cells from an HLA-A2-positive donor, or, in the case of the Tconvs, islet cells from an HLA-A2-positive donor or an HLA-A2-negative donor. Untransduced CD4+ T cells and anti-HLA-A2 CAR-transduced CD4+ T cells from the same donor were used as controls. Untransduced CD4+ T cells and anti-HLA-A2 CAR-transduced CD4+ T cells were not activated by HLA-A2-negative islet cells, while the anti-DPP6 CAR-expressing Tconvs were activated (FIGS. 4A-4C). HLA-A2-positive islet cells induced strong expression of the different activation markers on both anti-DPP6 CAR-expressing Tconv and Tregs, as well as on anti-HLA-A2 CAR-transduced CD4+ T cells (FIGS. 4D-4F, 5A-5C). Importantly, an increase in activation marker expression was only observed when CAR T cells were co-cultured with islets, demonstrating the absence of any tonic signaling. All together, these data demonstrated a robust and specific activation in vitro of anti-DPP6 CAR-expressing human T cells by primary human islets.

To further evaluate the in vitro functionality of the anti-DPP6 CAR-expressing human T cells, the engineered T cells were cultured in the presence and absence of human SBC for 48 hours before T cell activation was assessed by flow cytometry. The expression of activation markers (CD71, ICOS, CD25) by polyclonal and anti-DPP6 CAR-expressing CD4+ conventional T cells is shown in Table 1-5. The expression of activation markers by polyclonal and anti-DPP6 CAR-expressing CD4+ regulatory T cells is shown in Table 1-6. Thus, anti-DPP6 CAR expressing CD4+ Tconv and Tregs get specifically and robustly activated by SCB in vitro.

TABLE 1-5
Activation of CD4+ Conventional T Cells\hat{ }

| | | Clone 1 | Clone 2 | | Clone 1 | Clone 2 |
|---|---|---|---|---|---|---|
| | | DPP6 | DPP6 | | DPP6 | DPP6 |
| | Poly | CAR | CAR | Poly | CAR | CAR |
| | Tconv | Tconv | Tconv | Tconv | Tconv | Tconv |
| Marker | Alone | Alone | Alone | +SCB | +SCB | +SCB |
| CD71 MFI | 1109 | 307 | 3424 | 805 | 24990 | 32774 |
| ICOS MFI | 707 | 42 | 1511 | 695 | 383 | 4312 |
| CD25 MFI | 1911 | 1918 | 2914 | 1833 | 42586 | 25060 |

\hat{ }Poly Tconv data is the average of n = 2.
Clone 1 refers to Tconv expressing the 2hD6 CAR.
Clone 2 refers to Tconv expressing the 2hD123-A24V CAR.

TABLE 1-6
Activation of CD4+ Regulatory T Cells\hat{ }

| | Poly | Clone 2 | Poly | Clone 2 |
|---|---|---|---|---|
| | Tregs | DPP6 CAR Tregs | Tregs | DPP6 CAR Tregs |
| Marker | Alone | Alone | +SCB | +SCB |
| CD71 MFI | 4854 | 7537 | 2423 | 8709 |
| ICOS MFI | 1629 | 2070 | 2042 | 13755 |
| CD25 MFI | 12045 | 17176 | 17462 | 48972 |
| Foxp3 MFI | 1731 | 1960 | 2043 | 3808 |
| CTLA4 MFI | 845 | 921 | 742 | 1928 |

\hat{ }Clone 2 Tregs data is the average of n = 2.
Clone 2 refers to Tregs expressing the 2hD123-A24V CAR.

Next, the anti-DPP6 CAR-expressing human T cells were administered to mice in order to test whether the T cells would migrate to and be activated by human islets in vivo. Specifically, anti-DPP6 CAR-expressing CD4+ Tconvs (FIG. 6) or Tregs (FIG. 7) were injected into immunodeficient mice that had previously received human islet transplants from HLA-A2-negative and HLA-A2-positive donors, respectively.

Of the mice injected with Tconvs, a rapid and strong increase in glycemia was observed less than 10 days after T cell injection only in the group of mice that received anti-DPP6 CAR-expressing Tconvs (FIG. 6). Indeed, mice injected with polyclonal or anti-HLA-A2 CAR-expressing Tconvs remained normo-glycemic. These results demonstrate the capacity of anti-DPP6 CAR-expressing Tconvs to traffic in vivo to transplanted human islets, as well as their proper activation and function induced by the recognition of their target (DPP6) via the CAR.

In the Treg administration experiment, for more than 1 month after the CAR Treg injection the mice remained normo-glycemic. To ensure that this observation was not due to a rebound of mouse islets, a nephrectomy of the kidney transplanted with human islets was performed on all mice and glucose levels were monitored daily. Shortly after nephrectomy, a rapid and substantial increase in glycemia was observed in all the animals. This observation confirmed not only the lasting functionality of the transplanted human islets, but also the absence of toxicity of the injected CAR Tregs.

In parallel, the in vivo migration of the CAR Tregs was evaluated by bioluminescence. While the anti-HLA-A2 CAR-expressing Tregs accumulated within a few days in the kidney transplanted with the human islets, anti-DPP6 CAR-expressing Tregs took longer to do so. Indeed, the bioluminescence signal remained wide spread for more than one week, with most luminescence seen around the spinal cord and the brain tissue where mouse DPP6 is expressed. This demonstrates that while anti-DPP6 nanobody had not been reported to cross the brain blood barrier, anti-DPP6 CAR Tregs can cross into the central nervous system. The ability of anti-DPP6 CAR-expressing Tconvs to interact with mouse DPP6 in vivo was also assessed. Specifically, CAR-expressing Tconvs that also express luciferase were injected into mice and visualized using bioluminescent imaging in vivo or ex vivo in isolated tissues. While polyclonal and anti-HLA-A2 CAR Tconvs gave a brief signal in the spleen before vanishing, a bright and persistent signal around the tissues of the central nervous system was observed in the mice injected with anti-DPP6 CAR expressing Tconvs. When individual tissues were imaged, signal in the brain was detected only in the mice injected with anti-DPP6 CAR-expressing Tconvs, confirming the potential of anti-DPP6 CAR to cross-react with mouse DPP6.

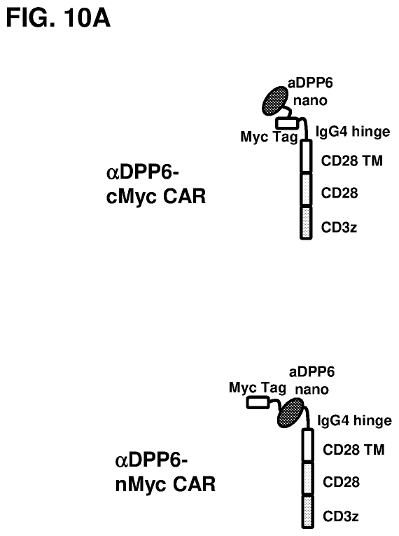

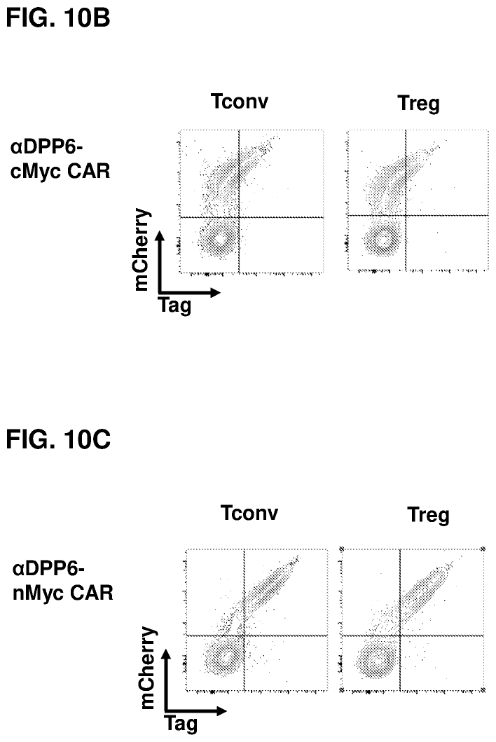

To determine the effect of the location of the Myc tag on detection of CAR expression on T cells, expression of a construct in which the Myc tag was placed at the N-terminal end of the 2hD123-A24V nanobody (nMyc) was compared to expression of a construct in which the Myc tag was placed at the C-terminal end of the 2hD123-A24V nanobody (cMyc). Schematic diagrams of the anti-DPP6-cMyc CAR and the anti-DPP6-nMyc CAR are shown in FIG. 10A (absent P2A and mCherry domains). CAR transduction efficiency and membrane expression were evaluated 5 days after transduction by assessing intracellular expression of mCherry protein and membrane expression of the Myc tag (FIGS. 10B-10C, and Table 1-7). Switching the Myc Tag from the C-terminus to the N-terminus of the nanobody improved the ability to detect anti-DPP6 CAR expression on transduced CD4+ Tconv and Tregs. Importantly, CD4+ Tconv and Tregs expressing either the anti-DPP6 nMyc CAR or the anti-DPP6 cMyc CAR were activated to similar levels when co-cultures with dissociated human islets for 48 hours (Table 1-8 and Table 1-9).

TABLE 1-7
Comparison of cMyc and nMyc CAR Expression on CD4+ T Cells\hat{ }

| | Tconv | | Tregs | |
|---|---|---|---|---|
| Marker | cMyc CAR | nMyc CAR | cMyc CAR | nMyc CAR |
| mCherry+ | 54 | 43 | 42 | 34 |
| Tag MFI | 258 | 2121 | 521 | 3189 |

\hat{ }Tregs data is the average of n = 2.

TABLE 1-8
Comparison of cMyc and nMyc CAR Activation
of CD4+ Conventional T Cells

| | cMyc CAR | nMyc CAR | cMyc CAR | nMyc CAR |
|---|---|---|---|---|
| Marker | Alone | Alone | +Islets | +Islets |
| CD71 MFI | 414 | 414 | 26970 | 42125 |
| ICOS MFI | 238 | 237 | 791 | 959 |
| CD25 MFI | 1460 | 1024 | 65793 | 75514 |

TABLE 1-9
Comparison of cMyc and nMyc CAR Activation
of CD4+ Regulatory T Cells

| | cMyc CAR | nMyc CAR | cMyc CAR | nMyc CAR |
|---|---|---|---|---|
| Marker | Alone | Alone | +Islets | +Islets |
| CD71 MFI | 1268 | 1536 | 6674 | 7792 |
| ICOS MFI | 1049 | 1225 | 2112 | 2392 |
| CD25 MFI | 53553 | 59105 | 87058 | 91230 |

In conclusion, anti-DPP6 CAR-expressing Tregs and Tconvs were generated, and determined to be capable of being specifically activated by human islet cells both in cell culture, and in vivo in a mouse model. Additionally, anti-DPP6 CAR-expressing Tregs and Tconvs were determined to be capable of being specifically activated by human stem cell-derived beta cells (SCB) in vitro.

Additional Sequences
(CD8a hinge)
SEQ ID NO: 32
TTTPAPRPPTPAPTIASQPLSLRPEACR
PAAGGAVHTRGLDFACD
(CD8a transmembrane)
SEQ ID NO: 33
IYIWAPLAGTCGVLLLSLVITLYC
(CD28 hinge)
SEQ ID NO: 34
IEVMYPPPYLDNEKSNGTIIHVKGKH
LCPSPLFPGPSKP

We claim:

1. A human regulatory T cell (Treg) engineered to express a dipeptidyl aminopeptidase-like protein 6 (DPP6)-reactive chimeric antigen receptor (CAR), wherein the Treg is CD4+, CD25+, CD127−/lo, and the CAR comprises an extracellular DPP6-binding domain linked through a hinge and a transmembrane domain to an intracellular domain comprising a costimulatory domain and an activation domain.

2. The human Treg of claim 1, wherein the extracellular DPP6 binding domain comprises a variable region of a DPP6-reactive nanobody, wherein the variable region comprises three complementarity-determining regions (CDRs) having amino acid sequences selected from:
(i) a CDR1 of SEQ ID NO:16, a CDR2 of SEQ ID NO:17, and a CDR3 of SEQ ID NO:18;
(ii) a CDR1 of SEQ ID NO:13, a CDR2 of SEQ ID NO:14, and a CDR3 of SEQ ID NO:15;
(iii) a CDR1 of SEQ ID NO:19, a CDR2 of SEQ ID NO:20, and a CDR3 of SEQ ID NO:21; and
(iv) a CDR1 of SEQ ID NO:22, a CDR2 of SEQ ID NO:23, and a CDR3 of SEQ ID NO:24.

3. The human Treg of claim 2, wherein the variable region comprises the amino acid sequence of SEQ ID NO:10, SEQ ID NO:9, SEQ ID NO:11, or SEQ ID NO:12, or an amino acid sequence sharing at least 90%, 95% or 99% identity with SEQ ID NO:10, SEQ ID NO:9, SEQ ID NO:11, or SEQ ID NO:12.

4. The human Treg of claim 2, wherein the hinge is an IgG4 hinge.

5. The human Treg of claim 4, wherein the hinge comprises an amino acid sequence sharing at least 90% identity with SEQ ID NO:3.

6. The human Treg of claim 2, wherein the transmembrane domain is a CD28 transmembrane domain.

7. The human Treg of claim 6, wherein the transmembrane domain comprises the amino acid sequence of SEQ ID NO:4, or an amino acid sequence sharing at least 90%, 95% or 99% identity with SEQ ID NO:4.

8. The human Treg of claim 2, wherein the costimulatory domain comprises a CD28 costimulatory domain.

9. The human Treg of claim 8, wherein the CD28 costimulatory domain comprises the amino acid sequence of SEQ ID NO:5, or an amino acid sequence sharing at least 90%, 95% or 99% identity with SEQ ID NO:5.

10. The human Treg of claim 2, wherein the activation domain comprises a CD3 zeta activation domain.

11. The human Treg of claim 10, wherein the CD3 zeta activation domain comprises the amino acid sequence of SEQ ID NO:6, or an amino acid sequence sharing at least 90%, 95% or 99% identity with SEQ ID NO:6.

12. The human Treg of claim 2, wherein the hinge, the transmembrane domain and the intracellular domain comprise the amino acid sequence of SEQ ID NO:25, or an amino acid sequence sharing at least 90%, 95% or 99% identity with SEQ ID NO:25.

13. The human Treg of claim 2, wherein the DPP6-reactive CAR further comprises an N-terminal signal peptide.

14. The human Treg of claim 13, wherein the N-terminal signal peptide comprises the amino acid sequence of SEQ ID NO:1, or an amino acid sequence sharing at least 90%, 95% or 99% identity with SEQ ID NO:1.

15. The human Treg of claim 2, further comprising a tag, optionally wherein the tag is a myc tag of SEQ ID NO:2 or a strep tag of SEQ ID NO:26, optionally wherein the tag is located on the N-terminal or the C-terminal side of the DPP6-binding domain.

16. The human Treg of claim 2, wherein the intracellular domain further comprises a self-cleaving peptide and a label C-terminal to the activation domain.

17. The human Treg of claim 16, wherein the self-cleaving peptide is P2A, and wherein the self-cleaving peptide comprises the amino acid sequence of SEQ ID NO:7, or an amino acid sequence sharing at least 90%, 95% or 99% identity with SEQ ID NO:7.

18. The human Treg of claim 16, wherein the label is a mCherry, and wherein mCherry comprises the amino acid sequence of SEQ ID NO:8, or an amino acid sequence sharing at least 90%, 95% or 99% identity with SEQ ID NO:8.

19. The human Treg of claim 2, wherein the DPP6-reactive CAR comprises the amino acid sequence of SEQ ID NO:28, SEQ ID NO:27, SEQ ID NO:29, or SEQ ID NO:30, or an amino acid sequence sharing at least 90%, 95% or 99% identity with SEQ ID NO:28, SEQ ID NO:27, SEQ ID NO:29, or SEQ ID NO:30.

20. The human Treg of claim 19, wherein the human Treg is FOXP3+, HELIOS+.

21. The human Treg of claim 20, wherein the human Treg has a FoxP3 promoter with a demethylated Treg-specific demethylation region (TSDR).

22. A method of treating or preventing type I diabetes in a human subject in need thereof, wherein the method comprises administering an effective amount of the pharmaceutical composition of claim 34 or claim 35 to the human subject.

23. A method of reducing hyperglycemia in a human subject in need thereof, wherein the method comprises administering an effective amount of the pharmaceutical composition of claim 34 or claim 35 to the human subject.

24. A method of preventing death of pancreatic beta-cells in a human subject in need thereof, wherein the method comprises administering an effective amount of the pharmaceutical composition of claim 34 or claim 35 to the human subject.

25. A method of treating or preventing an autoimmune disease of the nervous system in a human subject in need thereof, wherein the method comprises administering an effective amount of the pharmaceutical composition of claim 34 or claim 35 to the human subject, optionally wherein the autoimmune disease of the nervous system is autoimmune encephalitis or multiple sclerosis.

26. A method of treating or preventing a neurodegenerative disease in a human subject in need thereof, wherein the method comprises administering an effective amount of the pharmaceutical composition of claim 34 or claim 35 to the human subject, optionally wherein the neurodegenerative disease is selected from the group consisting of frontotemporal dementia, amyotrophic lateral sclerosis, progressive supranuclear palsy, Alzheimer's disease, and Parkinson's disease.

27. A method for the production of recombinant human regulatory T cells (Tregs) engineered to express a dipeptidyl aminopeptidase-like protein 6 (DPP6)-reactive chimeric antigen receptor (CAR), the method comprising:
a) culturing CD4+, CD25+, CD127−/lo human Tregs in medium comprising an activation agent under conditions effective in producing stimulated Tregs;
b) introducing a nucleic acid encoding a DPP6-reactive CAR comprising an extracellular DPP6-binding domain linked through a hinge and a transmembrane domain to an intracellular domain comprising a costimulatory domain and an activation domain into the stimulated Tregs under conditions effective in producing recombinant Tregs;
c) culturing the recombinant Tregs in medium comprising IL-2 under conditions effective in expanding an expanded population of recombinant Tregs; and
d) harvesting the expanded population of recombinant Tregs.

28. The method of claim 27, wherein step c) further comprises culturing the expanded population of recombinant Tregs in medium comprising an activation agent under conditions effective in producing restimulated Tregs.

29. The method of claim 28, wherein the activation agent comprises CD3 and CD28 agonists for cross-linking CD3 and CD28 of the Tregs, wherein the CD3 and CD28 agonists comprise monoclonal antibodies or fragments thereof coupled to a multimerization agent.

30. The method of claim 28, wherein the activation agent comprises a CD28 superagonist antibody in the absence of an anti-CD3 antibody.

31. The method of claim 27, wherein the CD4+, CD25+, CD127−/low T cells of step a) are isolated by fluorescence-activated cell sorting (FACS) or magnetic-activated cell sorting (MACS) from a lymphocyte-containing biological sample obtained from a human subject.

32. The method of claim 31, wherein the lymphocyte-containing biological sample is selected from the group consisting of whole blood, a leukapheresis product, and peripheral blood mononuclear cells.

33. The method of claim 31, wherein the lymphocyte-containing biological sample is either fresh or cryopreserved and thawed after being obtained from the human subject.

34. A pharmaceutical composition comprising from 10⁷ to 10¹¹ of the human Tregs of claim 2.

35. A pharmaceutical composition comprising from 10⁷ to 10¹¹ of the human Tregs produced by the methods of claim 27, and a physiologically acceptable buffer.
